(19) United States
(12) Patent Application Publication
(10) Pub. No.: US 2010/0280763 A1
(43) Pub. Date: Nov. 4, 2010

(54) BIOINFORMATIC TRANSACTION SCHEME

(76) Inventor: Dennis Sunga Fernandez, Atherton, CA (US)

Correspondence Address: FERNANDEZ & ASSOCIATES, LLP, P.O. BOX D, MENLO PARK, CA, 94026, US

(21) Appl. No.: 12/835,698

(22) Filed: Jul. 13, 2010

Related U.S. Application Data
(62) Division of application No. 11/776,539, filed on Jul. 11, 2007.
Application No. 11/776,539 is a division of application No. 09/435,504, filed on Nov. 6, 1999.

(51) Int. Cl. G06F 19/00; G01N 33/50

(52) U.S. Cl. 702/19

(57) ABSTRACT

Secure network transaction system obtains user-authorized genetic term or bioinformatic profile, and transacts online service according to genetically-based user medical or other risk determined therefrom. Insurance policy, promotional offer, or other service may dynamically address genetically-based condition. Bioinformatic data classifies user per personal mask which filters subset of user genetic sequence. Risk profile may be calculated according to actuarial statistics, genetics and/or heredity using non-discriminatory rules specified for users in temporal or jurisdictional groups. User transactions are modifiable according to bioinformatic data representing genetically-based risk increase or decrease. Data is securely processed, modulated, and stored by network server for remote access and transaction using various portable user devices.

### CLAIM OF PRIORITY

This application is a divisional of U.S. patent application Ser. No. 11/776,539 filed on Jul. 11, 2007, which is a divisional of U.S. patent application Ser. No. 09/435,504 filed on Nov. 6, 1999.

### FIELD OF INVENTION

Invention relates generally to bioinformatics, particularly to networked computer transactions using gene-related user data.

### BACKGROUND

Bioinformatics refers to an emerging discipline which combines biology and computer science. In this growing interdisciplinary field, various techniques associated with genetics and pharmaceuticals may be combined with computers, software, databases, networks, and other digital processing technologies. Increasingly gene-sequence databases and analysis tools are available widely through the Internet and other distributed computerized systems to automate certain promising areas of drug discovery, particularly identification, screening, and prediction of target gene and protein structure and function.

Present bioinformatics systems and processes, however, are highly data intensive, sometimes processing billions of pieces of personal genetic data, much of which may be very confidential in nature. Accordingly, there is need for improved scheme to manage voluminous bioinformatics data, as well as any sensitive transactions related thereto.

### SUMMARY

Invention enables secure bioinformatic-based transaction, whereupon on-line service is provided according to voluntary genetic profile. For example, network message pertains to insurance policy, promotional offer, or other personalized service, dynamically considering medical or other genetic-based risk determined from confidential user profile. Bioinformatic classification of personal risk profile is authorized using logical masking procedure to filter effective user subset of reference gene sequence or related structure.

Optionally, user risk profile is calculated using actuarial statistics, genetics and/or heredity per non-discriminatory rules specified temporally or jurisdictionally. Transaction terms are modifiable dynamically in response to profile risk increase or decrease. Secure network server processes, encodes, and stores data for remote access and transaction by portable user devices.

### BRIEF DESCRIPTION OF FIGURES

FIG. 1a is a system network diagram for implementing present invention; FIG. 1b is a flow chart of steps for implementing one or more aspects of present invention; FIG. 1c is a functional block diagram of a user module for implementing one or more aspects of present invention.

FIG. 2 is a diagram illustrating data structure according to one or more aspects of the present invention.

### DETAILED DESCRIPTION OF PREFERRED EMBODIMENT

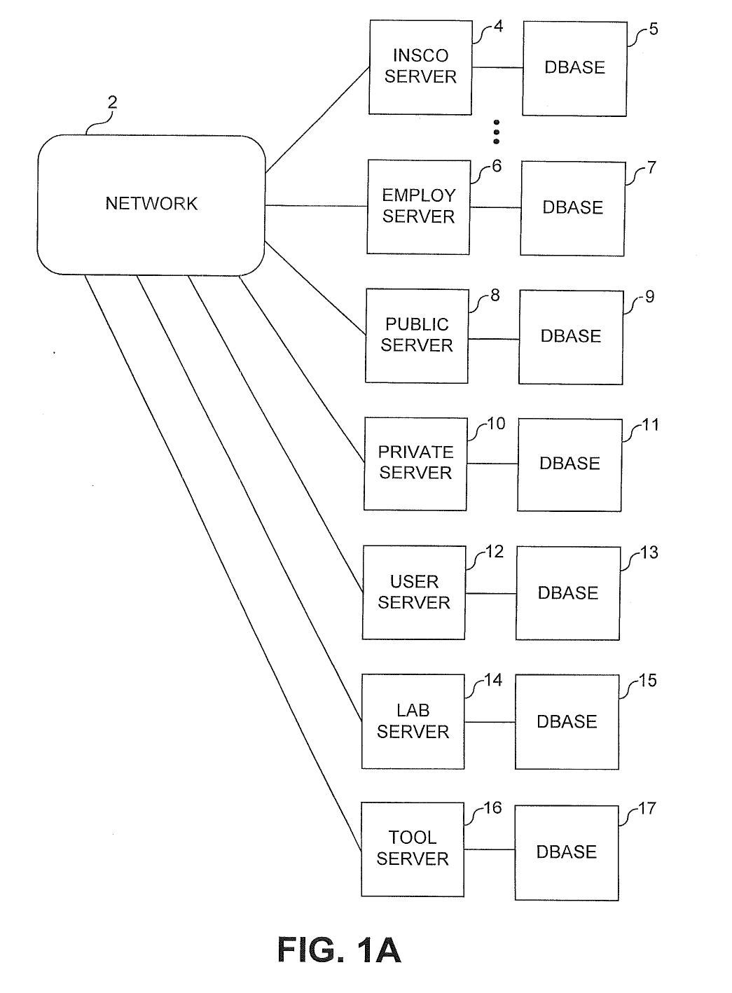

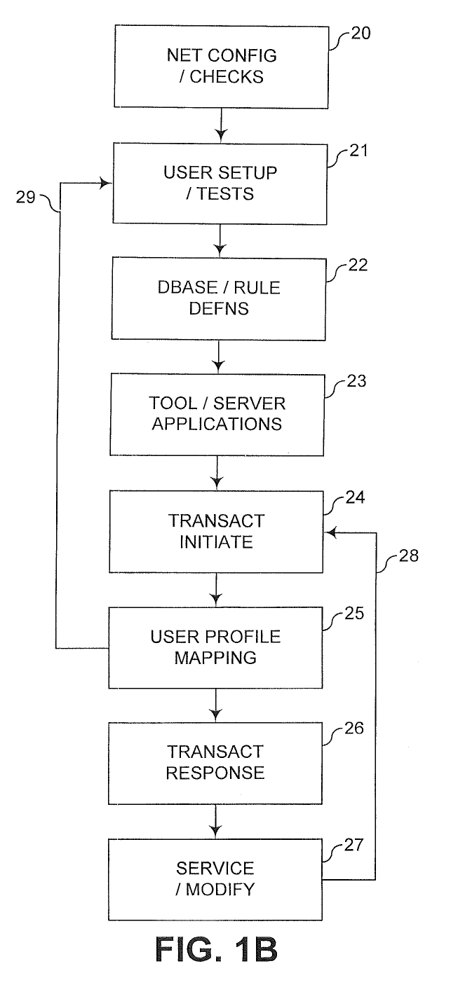

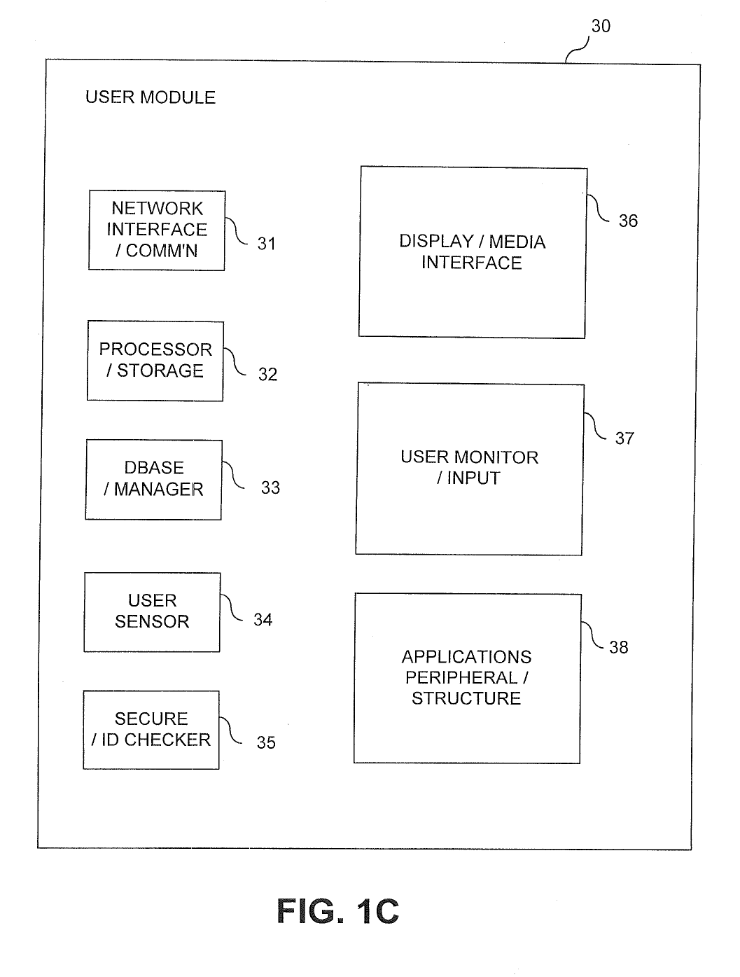

FIG. 1a diagram shows representative electronic signal and/or packet, cell, frame or other data format switching, bridging, routing, and/or digital network or related digital system architecture 2, which may be implemented using one or more interconnectable or coupled conventional or proprietary, wired and/or wireless, electronic and/or optical, terrestrial and/or satellite, local area network (LAN) and/or wide area network (WAN), or other network communications infrastructure equipment, electronics, software and/or related fixed or reconfigurable functionality. Network 2 serves generally for enabling local and/or remote electronic signal, file, or other data access, transfer, storage, or other applicable communications between network-accessible computers, processors, servers, appliances, or other addressable nodes.

As shown, network 2 may serve to interconnect nodes such as insurance or other service company server or compute device 4 having access to member terms, policy, or rule database or other storage repository 5; employer or other corporate server or compute device 6 having access to employee, rule, or application criteria database or other storage repository 7; public or other governmental server or compute device 8 having access to rule, discriminatory criteria, recommended sequence mapping segments and heuristics, genome sequence, or restricted classes database or other storage repository 9; private or other secure server or compute device 10 having access to personal reference sequence or profile, specialized services, or rule database or other storage repository 11; user or other client server or compute device 12 having access to personal reference sequence or profile, transaction account records, rules, specialized services, or sensor information database or other storage repository 13; laboratory or other test facility server or compute device 14 having access to personal reference sequence or profile, lab test sequencing results, or rules database or other storage repository 15; and tool or other automated applications server or compute device 16 having access to analysis software, specialized applications, or simulation programs and models database or other storage repository 17.

Each compute server facility 4, 6, 8, 10, 12, 14, 16 may operate independently or cooperative processing function effectively to distribute compute loading and data storage across scalable network resources.

Preferably, each such server is configured to run one or more conventional operating systems and programming languages and utilities, such as Windows, fortran, Unix, Linux, C/C++, perl, corba, cgi, etc.; one or more object-oriented or relational database management system to enable homogenous or heterogeneous data format and access, such as sql format; network communications interface management utility to enable apparently seamless file transfer and access, such as file transfer protocols, electronic mail, so-called htm/xml/java and other media format for web browse and on-line transaction and commercial access.

Generally, in a secure network configuration preferably according to the present automated transaction process and/or system, one or more personal or unique bioinformatic value, genetic term, DNA (deoxyribonucleic acid) sequence, folding structure, or subset thereof, or other biologically, hereditarily, or genetically identifiable or classifiable data associated with one or more user, participant, client, or other designated person or associated being is determined, provided, accessed, generated, calculated, processed, computed, or otherwise obtained.

Preferred bioinformatic value or genetic term is accessed, provided or generated as digital or alphanumeric data structure, including one or more user identifier field, and genetic sequence subset, mask, screen, or, filter field, effectively such that user reference sequence is processable securely for authorized transaction using sequence subset or genetic mask to qualify or otherwise evaluate participating user. Identifier may include partial or complete user social security number or other unique, random, or signature code.

Additionally, such data structure may include application-specific transaction control and payload fields, depending on user-authorized transaction basis. Optionally, data structure may be provided digitally in representative electronic signal form which may be encoded, compressed, transmitted, stored, received, and decoded, according to one or more secure signal or data modulation scheme, as spread spectrum, or other time/frequency/code-division multiple access (T/F/CDMA) scheme.

In this encoded/decoded manner, bioinformatic data, personal genetic sequences, or subsets thereof may be selectively accessed and communicated from network databases with reduced risk of publicly revealing confidential data, particularly by using randomized key coding and frequency-hopping scheme for spread spectrum communications and signaling techniques, as specified in any applicable published industry-standard modulation specifications, which documents are hereby incorporated by reference as appropriate. Accordingly, user transmission of confidential bioinformatic data as encoded signals between servers through network 2 is accomplished with reduced risk of public exposure.

Further, generally in accordance with preferred bioinformatic transaction scheme, transaction, communication, operation, negotiation, solicitation, or other automated service interaction with, related to, or associated with the user is initiated, triggered, modified, consummated, delivered, authorized, terminated, or otherwise responsively conducted according to, depending on, or upon condition of one or more of the bioinformatic values, particularly user risk analysis or condition determined therefrom.

Preferably, bioinformatic value represents, or may be processed to determine or otherwise generate indication, propensity, probability, likelihood, susceptibility, vulnerability, inclination, risk, certainty, or other deterministic or statistical metric of particular or identified user having or developing a genetically-based or related condition, such as one or more medical, genetic, mental, emotional disease or other condition, which is known, indicated, published, or suspected according to one or more specified rule set, database, mapping criteria, approved table, or other applicable heuristic or algorithm for logically, structurally, or functionally linking given bioinformatic value to particular condition, set thereof, or probability of such condition(s) occurring.

Thus, in accordance with one or more aspects of the present scheme, bioinformatics-based transaction may serve remotely, securely and/or automatically to provide or enable provision of one or more users with one or more insurance or other liability policy or risk service, or promotional offer, multi-media audio/visual message, competitive bid, or other electronic communication, to cover or otherwise contemplate the occurrence or non-occurrence of one or more genetically-based or linked condition or personal consequences related thereto. For example, in on-line auction fashion, competing insurance companies may offer separate rate bids to user according to common bioinformatic value, as well as same or different actuarial table or risk formula.

In particular, bioinformatic value or genetic term as used herein may refer to or mean uniquely referenceable or personally identifiable data structure, alphanumeric or text string, electronic signal, or other representative digital information for classifying or otherwise processing as described herein of the user, preferably according to volunteered, permitted, or user-authorized mask, screen, filter, or other logical criteria effectively for defining, recognizing, identifying, or otherwise generating one or more subset or sequence portion of a more complete, reference, or generalized genetic sequence associated with the user or other reference entity.

Hence, for example, deterministic or predictive value of user being subject or likely to contract genetically-based condition or disease is determinable from bioinformatic value or genetic term. Thus, in an automated relationship, one or more parameter, term, condition, or other of specified classification for applicable user transaction(s) depends directly or indirectly on such bioinformatic, genetic, or predictability value.

In a simplified embodiment, predictive approach according to user-provided bioinformatic data is accomplished in evaluative software executed by insco server 4 which initially qualifies genetic term as valid user sequence segment(s) that contains generally recognizable code such as CGAT etc. Preferably, user segments are provided to correspond with pre-specified segments associated with specific sequence locations for evaluating designated classes of genetic-based conditions. Then, such software further aligns one or more user segments against entire or portion of general reference sequence corresponding to accepted standard genome map.

In this alignment process which compares user genetic terms against known sequence and associated conditions, server software may now evaluate actual personal genetic data that uniquely, albeit partially, describes bioinformatic profile of transaction participant. Meanwhile, user still effectively masks or screens other non-volunteered, presumably more confidential or less essential for transaction qualification, sequence segments from outside party review.

Such software may then generate comparison, mapping, analysis, or other evaluation results to indicate how such user segment revelation indicates or suggests likelihood or risk of having or developing certain genetically-based conditions that are indicated by matching certain sequence terms or groups thereof according to one or more predefined rules or heuristics.

One or more heuristic or rule may be provided similarly to one or more actuarial or risk table or transmitted in electronic form as a computational model following one or more high-level programming or spreadsheet language, such as C/C++ or other database management syntax. Further, such heuristic or rule may provide numerical or statistical instructions or groupings to assign or calculate one or more risk profile values to one or more user applicants according to individual characteristics, such as age, sex, smoker status, marriage status, prior medical history, etc.

Also, such heuristic or rule may allow applicable transaction evaluation software automatically to assign or compute one or more risk profile values to one or more user applicants according to pre-specified genetic mapping table or formula, particularly determining such risk profile value according to the presence or absence of one or more matching or non-matching genes at designated locations, sequence segments, or sets thereof, whereupon such designated locations, segments, or sets thereof, correspond and indicate a certainty, likelihood, unlikelihood or other predictive value associated with one or more genetically-based condition, medical disease, or other related factor.

Hence, users may permit screening, masking, filtering, or other secured approach to recognize or identify certain differentiated or identical genetic elements or functional structures related to, as a subset of, or otherwise comparable to a personal sequence file, such as a more complete general human genome or other personal reference sequence definition.

Additionally, such bioinformatic data or genetic term may be based on an established or calculated statistical or actuarial table or other database and genetic or heredity profile associated with the particular user or set thereof. Bioinformatic values or genetic terms may be determined by or through one or more network-accessible servers, and such values or terms are stored confidentially in one or more local or remote database associated therewith.

Processing of bioinformatic value or genetic term for enabling transaction with one or more user is performed dynamically in real-time according to one or more rule set applicable to one or more users assigned, classified, or otherwise provided in one or more temporal or jurisdictional grouping or category, preferably on non-discriminatory basis or other equitable threshold or fairness-based criteria among equivalently qualified or classified group members. In certain cases, bioinformatic value processing may indicate identical genetic terms, suggesting possible fraudulent sample data, sequence clone or twin matching, or other alert state to be reported and investigated.

Preferably, one or more public servers 8 provide updated database 11 containing acceptable or non-discriminatory sample sequence segments or index (i.e., so-called SNP) for defining user bioinformatic screening values, for example, resulting from ongoing, reliable, quality medical and scientific genetic research. Hence, in this organized screening scheme, various on-line transaction and other service providers may conduct more predictable analysis and evaluation of potential customers and service applicants, as described herein.

For example, one or more user or transacting servers may generate a potential discrimination-violation or other representative signal indication or report upon comparing equivalently profiled bioinformatic data for multiple user applicants for a given transaction offer, but one or more user applicant(s) is provided substantially advantageous transaction terms or policy rates over other user applicants, particularly where genetically-based difference between such advantaged vs. disadvantaged applicants substantially arises in non-permitted classifications according to specified rule set or heuristics, such as racial or ethnic character.

Optionally, one or more sequence segments may be designated by transaction processing rule set or heuristics to be blocked or otherwise disregarded from consideration for transactional risk analysis, otherwise, detection of such restricted analysis may result in discrimination indication, as described herein.

Optionally, one or more sets of multiple bioinformatic values and/or genetic terms which are associated with one or more users are determined, modified, tiered, ranked, or otherwise generated accordingly. Thus, present or previous transaction with specified user(s) according to prior or initial bioinformatic values may be modified correspondingly, for example, when newly-provided bioinformatic value represents increase or decrease of likelihood or risk of given user having or developing certain genetically-based condition.

When same or substantially equivalent bioinformatic value or genetic term is determined or associated with multiple users, corresponding transaction or other operation applicable with each user occurs confidentially, preferably processed separately according to each user bioinformatic value or genetic term on effectively non-discriminatory basis.

Preferably, network client or device associated with particular user or group thereof may process or couple to network 2 for interactive access and transaction therewith. For example, bioinformatic value or genetic term may be generated for one or more transaction operations in secure authenticated process by implantable or portable user device. Optionally, user account or repository provided in, or accessible to, user device is updated to identify, meter, or otherwise record billing charges, amount, frequency, quality, or other factor or account credits regarding certain or each user transaction or related network activity.

It is contemplated generally herein that user server or device module 12 may be implemented in various network-accessible or stand-alone personal computers, engineering workstations, personal digital assistants, processors, microcontrollers, servers, network appliances, or other addressable nodes, which provide storage and processing function.

Preferably, user device 12 includes one or more memory circuits or database software structure 13 for storing bioinformatic value or genetic term associated with one or more user, and microprocessor for securely controlling access to stored values and terms through network 2. Device microprocessor may enables secure access and transaction between servers 4, 6, 8, 10, 12, 14, 16. In particular, microprocessor may determine, flag, monitor, alert, or otherwise signal specified transaction conditions, such as unsecured access, multi-user transaction, same bioinformatic value condition, rule-violation transaction discrimination, etc.

FIG. 1b flow chart shows network 2 operational steps generally for automating bioinformatic transaction, whereby on-line or interactive digital service or other electronic messaging is secured using user-released genetic profile or subset.

Initially, network 2 is configured 20 to couple various addressable compute nodes 4, 6, 8, 10, 12, 14, 16, including associated corresponding heterogeneous or homogeneous databases 6, 7, 9, 11, 13, 15, 17 for appropriate client-server or peer-peer communications, control, and file-transfer relationships. Preferably, each server 4, 6, 8, 10, 12, 14, 16 coupled thereto is checked for secure authorization and appropriate participation or file access levels. As necessary to support and scale for increased network transaction load, additional servers and database may couple and be registered as users or service providers for one or more probable or qualified transaction groups.

One or more client or user 12 node may then be set-up 21 as well for network configuration and subscription for one or more specified network transaction categories or access groups. User device and software testing may be performed remotely for current calibration. Preferably, one or more network server provides transaction management control and overall servicing to coordinate messaging between transaction providers and various active or possible user devices coupled thereto.

One or more databases 5, 7, 9, 11, 13, 15, 17 may then be recognized, initialized, updated, or otherwise defined 22 as part, currently or potentially, for one or more specified network transaction categories or access groups. Heuristics or other rule sets as well are recognized, initialized, updated, or otherwise defined within one or more such databases for network access.

Preferably, any applicable bioinformatic or biogenetics-related database, heuristic, or rule format, specification, and interface or access requirements, for example, as used in conjunction with electronic data, signal, file or network transfer and communication, which complies with one or more published or industry-adopted standards or syntax, as well as conventional extensions thereof, are hereby incorporated by reference.

One or more software programs, application-specific programs, automation tools, or other transaction code are recognized, initialized, updated, or otherwise defined 23 as part, currently or potentially, for one or more specified network transaction categories or access groups.

One or more transaction or other operational message is transmitted or initiated 24 between client or user server and one or more other server accessible thereto through network 2. For example, representative initial message may be provided through network browser or other applications screen to introduce, advertise, remind, suggest, alert, or otherwise relate to medical or health insurance policy, marketing promotional offer for certain merchandise, or other personalized service communication.

In accordance with one aspect of bioinformatics-based transaction model, user profile or genetic risk mapping 25 occurs, dynamically determining or analyzing medical or other genetic-based exposure, for example, by comparing confidential user data or volunteered sequence subset to specified actuarial tables, heredity background and propensity, transaction pre-qualification rule set for assigning one or more user risk determinations to corresponding present or absent genetic sequence or other heuristic analysis tools to predict or calculate user likelihood of having or being predisposed to one or more genetically-based conditions.

In particular, bioinformatic classification of personal risk profile is securely authorized using logical masking or screening procedure to filter effective user subset of reference gene sequence or related structure, relatively efficiently without necessarily identifying, transmitting, or storing complete or significant portion of content of confidential user genetic sequence data.

Selective segment revelation, preferably limiting disclosure deliberately by user only to personal gene sequence locations associated with transaction evaluation and related personal risk, particularly where established research data confirms high correlation between certain sub-segment sequencing and occurrence or likelihood of certain medical or disease conditions, significantly reduces exposure of confidential bioinformatic data, and general data size for transmitting, storing, and analyzing such data, since more complete personal gene sequence or genome is not disclosed or processed.

Hence, upon user profile mapping 25, secure network transaction system obtains user-authorized genetic term or bioinformatic profile, and responsively transacts 26 on-line service or other transactional operation, for example, according to genetically-based user medical or other risk determined therefrom.

As further illustration, certain insurance company transaction server 4 may determine for particular user applicant that provided and authorized bioinformatics value, as determined from selected sequence masking of reference genome results in 0-5% likelihood or risk profile that such candidate insuree be exposed to serious health condition or diseases (such as heart problem) within given temporal range of 5-year policy and California jurisdictional residence, and additionally that such applicant's hereditary and other non-genetic profile (e.g., non-smoker, no family cancer) does not significantly contribute to calculated risk.

Advantageously, insurance company may benefit from having more accurate determination of applicant risk profile, while applicant benefits as well from potentially lower policy rates due to favorable bioinformatic value submission.

User transactions and/or corresponding services are modifiable 27 according to bioinformatic data representing genetically-based risk increase or decrease. Data is securely processed, modulated, and stored by one or more network server for remote access and transaction using various portable user devices. Insurance policy, promotional offer, or other service may dynamically address and be modified accordingly based upon genetically-based condition in virtually real-time.

After current transaction or servicing is completed 27, new transaction messaging operations 24 may be re-started 28 to accommodate new or revised transaction requirements. Various transaction applications may continue with one or more related messaging or signaling between transaction server and user, as appropriate to conduct particular transaction.

Moreover, after user profile mapping 25 is completed, user setup and testing 21 may be restarted 29 to accommodate new or revised user or client network participation. Accordingly, present bioinformatics-based transaction scheme may continue on ongoing basis, responding dynamically to user requests, modifications, or signals.

As described herein, bioinformatic data classifies user per personal mask which filters subset of user genetic sequence, and risk profile is calculated according to actuarial statistics, genetics and/or heredity, preferably using non-discriminatory rules specified for users in temporal or jurisdictional groups. Temporal grouping may be according to age, relevant time/day/week/month/year etc. Jurisdictional grouping may be according to address/community/city/state/country etc.

FIG. 1c diagram shows preferred functional configuration of user module 30, which may include network communications interface 31 for coupling user module to network 2 for remote signaling and data access; processor and storage 32 for computing and storing digital instructions, signals, and data; database manager 33 for controlling and managing read, write, modify, and delete access to database 13; one or more user mixed-signal sensors 34 for electronically detecting one or more biometric or bioinformatic values, or conditions associated with user; secure identification checker 35 for authenticating correct user via text, signature, voice, retinal, fingerprint, or other identifiable objective input; visual and/or audio display screen/speaker media output interface 36 for communicating transaction messages with user; user monitor input 37 for monitoring user video and/or sound input for communication therewith; and applications peripheral and related interface or bus or signaling structure 38 for coupling user module 30 to other specific or general digital or analog devices for communication or signaling therewith. It is contemplated herein that user device may be more simplified, including merely storage and processing function to handle secure bioinformatic data access.

As described herein, user module 12, 30 is embodied preferably in any portable network-accessible device which may store user bioinformatic data and control network access to stored data.

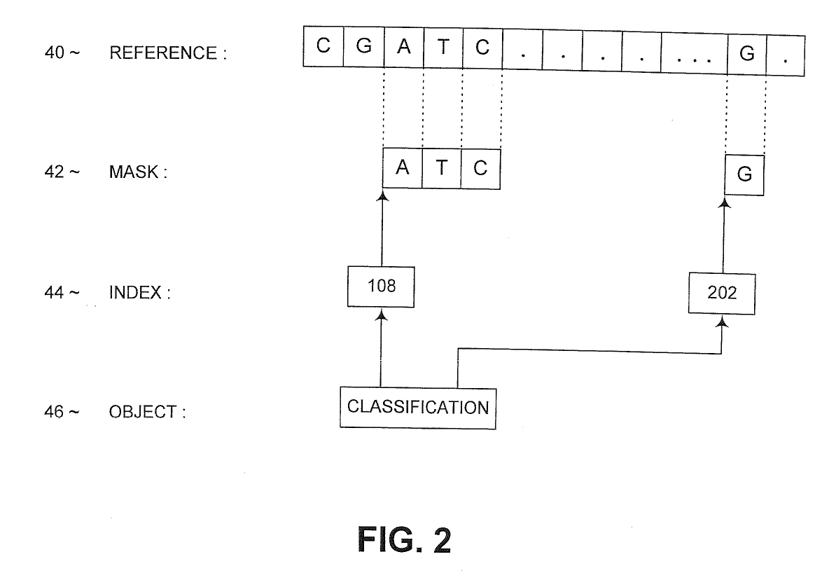

FIG. 2 diagram shows sample bioinformatic data structures, including reference sequence 40 (partial), mask subset 42 as well as indexing flags 44 aligned therewith, and classification object 46. Optionally, flags 44 may correspond with one or more so-called single nucleotide polymorphisms (SNPs), and thereby associate, mark, link, map or otherwise indicate user propensity for illness or particular disease or combination thereof.

In particular, data structure represented by mask 42, or representative or functional indication, thereof is preferably provided or released by user to authorize or otherwise permit network transaction activity, and may designate one or more bioinformatic value or genetic term which uniquely references or personally identifies user risk classification or other genetically-based grouping.

In this more secure methodology, predictive value of user being subject or likely to contract genetically-based condition or disease is determinable from bioinformatic value or genetic term. Thus, in network computer relationship, one or more parameter, term, condition, or other of specified classification for applicable user transaction(s) depends directly or indirectly on such bioinformatic, genetic, or predictability value accessible thereto electronically.

Hence, mask 42 and/or index 44 effectively enables simplified (e.g., positive exposure, or negative block) screening, filtering, or other approach to recognize or identify certain differentiated or identical genetic elements or functional structures related to, as a subset of, or otherwise comparable to a personal sequence file, such as a more complete general human genome or other personal reference sequence definition.

In preferred embodiment, it is contemplated that one or more user or other reference human gene sequence or genome set is pre-determined and stored as a relatively large sequential file in one or more database accessible via network 2. However, in accordance with one or more aspect of present invention, user may confidentially test using conventional gene sequencing methods his or her biological material, such as hair, blood, etc., to obtain personal genetic sequence or selected segments thereof.

For example, user-disclosed bioinformatic data or related genetic values may be selectively revealed or authorized, depending on applicable transaction server application, such as medical screen segmentation for insurance company considering future health risk, and non-medical screen segmentation for potential employer considering future management capabilities. Confidential laboratory data may be stored or accessed, in full sequence or partially masked, with proper authorization from database 15 of lab server 14.

As discussed herein, one or more selected portions of such personal sequence may be defined and released preferably on confidential basis as logical screen or access key to expose or block gene sequence data from other-party transaction scrutiny. Such limited exposure may adequately enable transaction party to define or modify transaction terms, such as personal medical risk to specify insurance policy rates.

Foregoing described embodiments of the invention are provided as illustrations and descriptions. They are not intended to limit the invention to precise form described. In particular, Applicant contemplates that functional implementation of invention described herein may be implemented equivalently in hardware, software, firmware, and/or other available functional components or building blocks. Other variations and embodiments are possible in light of above teachings, and it is thus intended that the scope of invention not be limited by this Detailed Description, but rather by Claims following.

What is claimed is:

1. A handheld bioinformatic apparatus comprising:
one or more bio sensors for obtaining and processing a DNA and protein sample from the user or other designated person,
a database for storing genomic, proteomic, and phenotypic information about the user or other designated person,
an interface for enabling access to one or more network servers to provide secure transactions, enable effective disease risk assessment, disease diagnosis, and to predict possible responses to medical treatment for the user or designated person,
wherein a predictability value of the user being subject to disease is determinable from the DNA and protein sample obtained from the user or other designated person, a parameter of the disease risk assessment, disease diagnosis and medical treatment response prediction being dependent on the predictability value.

2. The handheld bioinformatic apparatus of claim 1 wherein:
one or more bio sensors monitor nucleic acid concentration by detecting a change of pH resulting from proton release when the amplification of a user or designated person's DNA proceeds beyond a threshold number of PCR cycles, thus overcoming the buffering capacity of the bio sensor's sample buffer, thereby enabling the generation of an electrical output signal in response to change of pH and its detection via an electrical output signal.

3. The handheld bioinformatic apparatus of claim 1 wherein:
one or more bio sensors comprise an ion-sensitive field effect transistor (ISFET) having a sensing surface exposed to the sample and arranged to generate an electrical output signal in response to change of pH at said transistor surface and means for detecting an electrical output signal from said ISFET.

4. The handheld bioinformatic apparatus of claim 1 further comprising:
a microfluidic device in which one or more chambers or channels to receive the DNA and protein sample are in or on a substrate chosen from the group of silicon, glass, polymers or ceramics, and one or more pH-sensitive ISFETS are arranged for monitoring of nucleic acid amplification in said chambers or channels.

5. The handheld bioinformatic apparatus of claim 1 wherein:
the DNA and protein samples obtained from the users or other designated persons comprise a set of single nucleotide polymorphisms (SNPs) and target proteins associated with heart disease, thereby enabling effective heart disease diagnosis and prediction of possible responses to medical treatment for heart disease.
